(19) United States
(12) Patent Application Publication
(10) Pub. No.: US 2008/0033396 A1
(43) Pub. Date: Feb. 7, 2008

(54) VASCULAR SHEATHS AND METHODS FOR THEIR DEPLOYMENT

(75) Inventors: Christopher Danek, San Carlos, CA (US); Robert S. Behl, Palo Alto, CA (US); Julia Rasor, Los Gatos, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Percutaneous Systems, Inc., Mountain View, CA (US)

(21) Appl. No.: 11/777,768

(22) Filed: Jul. 13, 2007

Related U.S. Application Data
(60) Provisional application No. 60/821,002, filed on Aug. 1, 2006.

(51) Int. Cl. A61M 25/01

(52) U.S. Cl. 604/508; 604/163

(57) ABSTRACT

A percutaneous luminal access system comprises a thin-walled, collapsible sheath, an introducer, a hemostatic valve, and an access catheter. An introducer may comprise either a pusher tube or an elongate member, or where the introducer is used to axially advance the sheath into a blood vessel or other target lumen. A hemostatic valve may be connected to a proximal end of the sheath, and the access catheter introduced through the hemostatic valve. Pressurized fluid may also be introduced through the hemostatic valve and delivered through a flow region around the catheter within the sheath and optionally through the catheter to the target luminal site.

### CROSS-REFERENCES TO RELATED APPLICATIONS

The present application claims the benefit of provisional U.S. Application No. 60/821,002 (Attorney Docket No. 021807-003400US), filed Aug. 1, 2006, the full disclosure of which is incorporated herein by reference.

### BACKGROUND OF THE INVENTION

1. Field of the Invention

The present invention relates generally to medical methods and apparatus. More particularly, the present invention relates to methods and systems for deploying collapsible sheaths for providing vascular access.

Access to a patient's vascular system is required under numerous circumstances. Access is often obtained using a catheter which is introduced percutaneously so that a distal end of the catheter is in the vasculature while a proximal end remains outside available for infusion, aspiration, and often for the delivery of other catheters and interventional and diagnostic devices. Of particular interest to the present invention, a variety of catheters are used to establish access over extended periods of days, weeks, and in some cases even months. Such catheters, commonly referred to as “in-dwelling” catheters, are typically used for accessing the venous system to permit the administration of fluids, drugs, nutrition, and for obtaining blood samples. Such in-dwelling catheters are also used for drawing and reinfusing blood in order to perform hemodialysis.

Although essential for a number of life-saving procedures, the presence of catheters in the vasculature over extended time periods is problematic. Catheters can partially occlude and limit blood flow. Moreover, catheters in the vasculature can be a source of clotting as well as emboli, and present other risks to the patient.

The use of “collapsible” catheters has been proposed for long-term vascular access. Such catheters are formed from a material with little or no hoop strength so that the body of the catheter will collapse under the pressure when not in use. The catheter lumens may be opened, however, by delivering a pressurized fluid to the catheter lumen and from there to the blood vessel.

One shortcoming of such collapsible vascular catheters is the inability to aspirate through the collapsible lumen. To overcome this shortcoming, it has been proposed to provide “hybrid” catheters having at least one non-collapsible lumen in combination with one or more collapsible lumens. See, for example, U.S. Pat. No. 4,406,656. While potentially advantageous, such hybrid catheters can never achieve the very low profile associated with completely collapsible catheters. Moreover, should any of the lumens in these catheters become occluded or otherwise inoperable, it is necessary to exchange and replace the entire catheter structure.

The placement of central venous catheters can present particular problems to the physician. Such catheters can be difficult to advance through tortuous anatomies, particularly when the anatomies are stenosised or otherwise diseased. Placement of a central venous catheter can also present a risk of infection as advancement of the catheter can carry in microorganisms. Additionally, exchange or replacement of the central venous catheters can be very difficult.

For these reasons, it would be desirable to provide improved and additional vascular access methods and systems which overcome at least some of the above difficulties. In particular, it would be desirable to provide catheter systems which are capable of collapsing to a very low profile while simultaneously providing the ability to aspirate from the vasculature. Such vascular access systems should be relatively easy to deploy, should minimize the risk of infection resulting from deployment, and should simplify exchange or replacement should any part of the catheter become occluded or otherwise defective. Such catheter systems and deployment methods should be suitable for a variety of purposes, including infusion, aspiration, deployment of interventional tools, deployment of diagnostic tools, performing hemodialysis, and the like.

2. Description of the Background Art

The use of an everting sleeve composed of thin, tensilized polytetrafluoroethylene for introducing catheters to body lumens is described in U.S. Pat. Nos. 5,531,717; 5,676,688; 5,711,841; 5,897,535; 6,007,488; 6,240,968; and EP605427B1. Collapsible catheters for low profile vascular access are described in U.S. Pat. Nos. 4,406,656; 4,738,666; 5,106,368; 5,569,219; 5,472,418; 5,618,267; 5,807,311; 5,827,243; 6,096,013; and 6,926,509. Other catheters employing everting sleeves for a variety of purposes are described in commonly assigned, copending application Ser. Nos. 10/794,337 (Attorney Docket No. 021807-000300US), filed on Mar. 5, 2004; 10/794,317 (Attorney Docket No. 021807-000400US), filed on Mar. 5, 2004; 10/886,886 (Attorney Docket No. 021807-000800US), filed on Jul. 7, 2004; 10,951,922 (Attorney docket No. 021807-000700US), filed on Sep. 27, 2004; 10/993,631 (Attorney Docket No. 021807-001300US), filed on Nov. 19, 2004; 11/223,886 (Attorney Docket No. 021807-002600US), filed on Sep. 23, 2005; 11/256,562 (Attorney Docket No. 021807-002700US), filed on Oct. 20, 2005; 11/346,606 (Attorney Docket No. 021807-001410US), filed on Feb. 1, 2006; and 11/367,084 (Attorney Docket No. 021807-003000US), filed on Mar. 3, 2006, the full disclosures of which are incorporated herein by reference.

### BRIEF SUMMARY OF THE INVENTION

The present invention provides methods and systems for accessing target locations within various lumens in a patient's body. While the methods and systems are particularly useful for accessing the vasculature, they will also find use for accessing other body lumens where chronic or long-term fluid exchange is necessary, for example to access the peritoneum for peritoneal dialysis. Other body lumens which may be accessed include the peritoneal cavity, the gastrointestinal tract, and the like.

The vascular or other target locations will most commonly be reached percutaneously. By “percutaneously,” it is meant that an access sheath and catheter will be introduced through a penetration formed in the skin and through underlying tissue to reach the target location. A variety of suitable percutaneous access techniques exist, including the Seldinger technique where the sheath and catheter are introduced over a guidewire without using a separate introducer sheath. More typically, when introducing the sheath to the vasculature, a modified Seldinger technique will be used where an introducer sheath has been placed using a guidewire, and the sheath and catheter are thereafter introduced over the guidewire through the introducer sheath. For access to other body lumens, such as the peritoneum, laparoscopic and other introduction methods, typically using a cannula, may be employed. Percutaneous access tracts may also be formed by “button” or “buttonhole” techniques. In such techniques, repeated percutaneous access through the same tract and tissue causes the tissue surrounding the tract to fibrose, and over time become resistant to healing. The fibrosed tract thus remains patent and available for percutaneous access in the methods of the present invention.

In a first aspect of the present invention, a method for establishing luminal access comprises percutaneously introducing a thin-walled, collapsible sheath into a body lumen. A catheter, usually non-collapsible, is then positioned within a lumen of the sheath where the size of the catheter and the size of the sheath are selected to leave an annular space or gap between an outside surface of the catheter and an inside wall of the sheath. In this way, the sheath provides a collapsible annular lumen (referred to as a “flow area”), which can be used to deliver fluids to the target location within the body lumen. When delivery is stopped, the sheath can collapse to a low-profile. The non-collapsible catheter provides a lumen for either aspirating from the target location or delivering other fluids to the target location. The catheter will typically have a diameter or width which is much less than that of the collapsible sheath (when fully inflated by fluid delivery pressure) so that the profile of the system as a whole is significantly reduced when the fluids are not being delivered through the collapsible sheath.

Moreover, since the collapsible sheath and non-collapsible catheter are separate components, they may be removed and replaced independently of each other should one of them fail (where failure may occur as a result of thrombosis, clotting-off, bacterial colonization, mechanical failure, or the like) or should a different sheath or catheter be desired (different catheters may be desired to provide for a different number or configuration of the lumens or for other purposes). In other instances, the non-collapsible catheter may be removed and exchanged or replaced on a regular basis with the collapsible sheath remaining in place to provide an access lumen to minimize trauma to the patient caused by the removal and replacement. The available replacement path also reduces the risk of infection of the blood vessel and tissue access route.

The relative size of the sheath and catheter may be selected to provide an annular lumen or flow area which is relatively large and can provide a low flow resistance path for introducing fluids into the vasculature or other body lumens. Typically, the cross-sectional area of the catheter will be less than 50% of the luminal area of the sheath, preferably being less than 33%. Frequently, the lumen of the sheath will be sized so that it can accommodate two or more non-collapsible catheters simultaneously. In this way, a new catheter can be introduced while a previously placed catheter is left within the sheath. The previously-placed catheter can then be withdrawn to provide for a catheter exchange while leaving at least one catheter in place at all times.

The thin-walled, collapsible sheath may be introduced into the body lumen in several different ways. In a first embodiment, a pusher tube may be used to advance and evert the sheath into the body lumen. The sheath will be initially stowed in a lumen of the pusher tube, with the sheath being pulled over a distal tip of the pusher tube as the pusher tube is advanced into the body lumen. Usually, the pusher tube will be advanced beyond a distal end of the sheath in order to detach the sheath from the tube. The pusher tube may then be pulled from the lumen of the sheath, leaving the sheath in place in the body lumen. Usually, the pusher tube will advanced over a guidewire into the body lumen, particularly when the body lumen is a blood vessel.

In a second embodiment, the sheath may be introduced using an elongate member which is attached to a distal end of the sheath. The elongate member, which may have a solid core (similar to a obturator, stylet, or wire) or may be a hollow tube, is released from the sheath after a distal end of the sheath has arrived at the target location in the body lumen. The sheath may be released, for example, by advancing and/or torqueing the elongate member while movement of the sheath is constrained in order to pull the member from the attachment point with the sheath. Alternatively, a separate detachment tool may be advanced over the elongate member in order to detach the sheath. Alternatively, electrolytic, chemical, mechanical, or other detachment mechanisms may be provided.

The elongate member may be disposed either within a lumen of the sheath or along side and parallel to the sheath, so the sheath is advanced. When the elongate member is a hollow tube (such as a catheter), the member may be introduced over a separate guidewire. When the elongate member has a solid core, it may be formed in a manner similar to a guidewire so that the elongate member can be advanced in a manner analogous to conventional guidewire advancement.

The methods of the present invention will, in some instances, be useful for placing a thin-walled, collapsible catheter even without subsequent positioning of an access catheter therein. In particular, the use of the pusher tube for everting introduction of the collapsible vascular sheath has been found useful. Similarly, the use of a tubular elongate member for advancing a collapsible sheath into the vasculature is also useful without subsequent placement of a catheter.

In many cases, however, the methods and systems of the present invention will be used for placing at least one access catheter within the thin-walled, collapsible sheath. In certain cases, the access catheter may be provided by the pusher tube which is used to initially advance the collapsible sheath. More commonly, however, the pusher tube or elongate member will be removed from the collapsible sheath, and the access catheter (which will typically be more flexible and comfortable than the member used to introduce the sheath) will then be advanced through the lumen of the collapsible sheath, typically being advanced beyond the distal end of the sheath. Usually, the sheath will include only a single lumen, but in other instances, a sheath having multiple lumens may be used. In those instances, the access catheter will typically be introduced through one of the sheath lumens, while the remaining sheath lumens are available for introducing fluids and/or additional catheters.

Typically, the sheath will have a luminal area which is significantly greater than the cross-sectional area of the access catheter. Because the flow area provided by the annular gap between the outside of the access catheter and inside of the sheath is proportional to the square of the sheath diameter, even a relatively small annular gap can provide substantial flow area. It is still preferred, however, to utilize access catheters having a relatively small diameter since the access diameters will typically be non-collapsible and will thus generally define the occlusive profile of the system when the sheath is collapsed. For example, the sheath will typically have an inner diameter in the range from 1 mm to 12 mm, usually from 1 mm to 5 mm, while the access catheter will have an outer diameter in the range from 0.5 mm to 6 mm, usually from 1 mm to 4 mm. The width of the annular gap between the access catheter and the sheath when fully expanded (and assuming they are placed coaxially) will typically be at least 0.5 mm, frequently being in the range from 0.5 mm to 2 mm.

In a second aspect, the present invention provides methods for introducing a thin-walled, collapsible catheter and a non-collapsible catheter specifically into a blood vessel. A guidewire is first placed in the blood vessel, and a pusher tube is advanced over the guidewire, where the collapsible sheath everts from a distal end of the pusher tube as the tube advances into the blood vessel lumen. Once in place, the pusher tube may be relied to provide an access catheter, or the pusher tube may be removed. When the pusher tube is removed, the sheath will usually provide a very large lumen (low flow resistance) device for the infusion of pressurized fluids, as generally described above. More typically, an access catheter will be introduced through the sheath and the combination of sheath and access catheter used for various combinations of infusion and aspiration.

In a third aspect of the present invention, an elongate member is advanced percutaneously into a lumen of the blood vessel. A distal end of a collapsible sheath is releasably carried into the blood vessel lumen by the member. The member is then released from the sheath after the sheath has reached a target location in the blood vessel. The member is removed from the blood vessel, leaving the sheath in place. An access catheter is then introduced through the sheath into the blood vessel, where an annular lumen is created between an outer surface of the catheter and an inner surface of the sheath. The combination of sheath and access catheter may be used for any of the purposes described above.

In a fourth aspect of the present invention, a system for establishing percutaneous access to a body lumen comprises a thin-walled, collapsible sheath having a lumen, a proximal connector, and a skin anchor. The system further includes an introducer coupled to the sheath and adapted to advance the sheath within the body lumen. The thin-walled, collapsible sheath will usually comprise a polymeric tube, and the polymeric tube will usually have a length in the range from 5 cm to 120 cm, typically from 10 cm to 60 cm, with an inner diameter in the range from 1 mm to 12 mm, typically from 1 mm to 5 mm, and also a wall thickness in the range from 0.01 mm to 0.05 mm. The polymer is usually a lubricious polymer and/or it may be lubricated on the surface. Exemplary polymers include polytetrafluoroethylene (PTFE), polyethylene (PE), perfluoroalkoxy (PFA), polyurethane (PU), perfluoromethylvinylether (MFA), perfluoropropylvinylether (PPVE). In the exemplary case, the polymer comprises tensilized PTFE/PPVE copolymer.

The exemplary polymeric tube sheath may be formed in a variety of ways. In some instances, it may be extruded to form a tubular structure having no seems. Usually, however, it will be formed from one or more flat sheets of polymer material where the sheath(s) are attached together along one or more axially oriented edges. For example, a single elongate sheet of material may be folded over so that overlapping edges may be joined to form a single axial seam. Alternatively, two or more polymeric sheets may be attached along their respective edges to form two or more axial seams. The resulting polymer tubes may be heat-formed or otherwise treated so that they collapse to form relatively flat structures in the absence of pressurization within their lumens. Alternatively, the polymer tubes could be configured to maintain some minimum degree of open lumen even when the tube is being forced closed by external blood or other luminal pressures.

The proximal connector on the thin-walled, collapsible sheath is typically a luer or other conventional medical device connector. The skin anchor may comprise any structure capable of adhering a portion of the sheath which lies external to the patient to the patient's skin. For example, a variety of suture wings, adhesive strips, and the like, are available for immobilizing catheters and similar devices on the skin.

The systems of the present invention may further comprise a hemostatic connector, such as a Touhy-Borst valve which can removably attach or connect to the proximal end of the sheath. Typically, the hemostatic connector will include an axial branch for receiving an access or other catheter which is to be passed coaxially through the sheath, and at least a second branch coupled or otherwise connected to an annular region between an outside wall of the catheter (one present in the sheath) and an inside wall of the sheath. Usually, the catheter will provide a non-collapsible lumen which is suitable for both aspiration and introductions of materials to the body lumen. The annular region created within the sheath is usually suitable for the introduction of pressurized fluids, where in the absence of a pressurized fluid the sheath will collapse on itself or on to the catheter (one present) in order to minimize the profile of the access system when indwelling in the blood vessel or other body lumen.

The introducer of the systems of the present invention may comprise a tube or other elongate member adapted to advance the thin-walled, collapsible sheath into the blood vessel or other body lumen. An elongate member will typically have a proximal end and a distal end, where the member may be positioned in the sheath and have a distal end frangibly connected to a location on the elongate member near its distal end. Such frangible connection will be capable of being broken or selectively detached in a variety of ways. Typically, the elongate member and sheath may be connected by adhesives, thermal bonding, the use of staples or other fasteners, or the like. Such connections may be broken by tensioning the connection, for example by axially advancing or torqueing the elongate member relative to the sheath. Alternatively, the connection may be broken by exposure to the luminal environment, for example by allowing the connection to dissolve in a fairly short period of time, or by the application of energy, such as electrical energy, ultrasonic energy, or the like.

The elongate member may take a variety of specific forms. In the simplest form, the elongate member may comprise a structure similar or identical to a vascular guidewire having a steerable tip and a proximal shaft. Usually, the sheath will be frangibly connected to the proximal shaft at a point proximal to the distal tip. Alternatively, the elongate member may comprise a tubular member having a central lumen adapted to receive a separate guidewire for introducing the elongate member and sheath to the body lumen. If the lumen to be traversed is not tortuous, the elongate member can be a solid stylet, typically fabricated from a polymer.

In an alternative embodiment, the introducer may comprise a pusher tube having a central lumen, where a distal portion of the sheath is disposed within the central lumen of the pusher tube so that the sheath will evert from the distal portion as the pusher tube is advanced through the body lumen while a proximal end of the sheath is held stationary. Usually, the pusher tube will be removed prior to introducing a separate access or other catheter. In some instances, however, the pusher tube may itself form an access catheter when left in place within the thin-walled, collapsible sheath.

The systems of the present invention may still further comprise an access or other catheter adapted to be introduced through the proximal connector and into the lumen of the sheath. Usually, the catheter will have an outside diameter which is smaller than an inside diameter of the sheath in order to leave an annular lumen when the catheter is positioned within the lumen of the sheath. For example, the catheter may have an outside diameter in the range from 0.5 mm to 6 mm, usually from 1 mm to 4 mm, and the inside sheath may have an inside diameter in the range from 1 mm to 12 mm, thus typically leaving an annular lumen having a width in the range from 0.5 mm to 2 mm.

### BRIEF DESCRIPTION OF THE DRAWINGS

FIG. 1 illustrates the components of a percutaneous luminal access system constructed in accordance with the principles of the present invention.

FIGS. 2A-2D illustrate advancement of a pusher tube to deploy a thin-walled, collapsible sheath in accordance with the principles of the present invention.

FIGS. 3A-3C illustrate use of a non-tubular elongate member for advancing and releasing the thin-walled, non-collapsible sheath of the present invention.

FIGS. 4A and 4B illustrate attachment of a hemostasis valve to the sheath of the present invention with subsequent introduction of an access catheter (FIG. 4B).

FIG. 5 illustrates a first method for forming a thin-walled, collapsible sheath in accordance with the principles of the present invention.

FIG. 6 illustrates a second method for fabricating a thin-walled, collapsible sheath in accordance with the principles of the present invention.

FIG. 7 is a detail illustrating a first technique for attaching an elongate member to a distal end of the sheath of the present invention.

FIG. 8 is a second detail illustrating an alternative approach for attaching an elongate member to a distal end of the sheath of the present invention.

FIGS. 9A-9D illustrate cross-sectional views of different combinations of access catheter and sheath, as taken along line 9-9 in FIG. 4B.

FIGS. 10A-10F illustrate introduction of an everting collapsible sheath using a pusher tube over a guidewire into a blood vessel with subsequent introduction of an access catheter and hemostatic valve, in accordance with the principles of the present invention.

FIGS. 11A-11C illustrate introduction of a collapsible sheath into a blood vessel using a non-tubular elongate member in the form of a guidewire.

### DETAILED DESCRIPTION OF THE INVENTION

Systems and methods according to the present invention provide minimally invasive and minimally traumatic access to blood vessels and other body lumens. Blood vessels may be arteries or veins, and other body lumens include the peritoneum for peritoneal dialysis, the gastrointestinal tract, and the like. Percutaneous refers to passing a sheath and optionally catheter of the system through the patient's skin into the blood vessel or other body lumen beneath a layer of tissue under the skin.

Systems of the present invention will comprise at least the sheath and an introducer for positioning or advancing the sheath through the skin penetration to the target body lumen. As the sheath will often be left in place for extended periods of time, often days, weeks, or even longer, the sheath will preferably have an anchor or attachment to the skin. The sheath will also usually include a luer or other connector at or near its proximal end to permit attachment to a fluid source, a hemostatic valve (for the introduction of access catheters), or the like.

Once in place, in the absence of internal pressurization, the sheaths will collapse in response to blood or other external luminal pressure so that the sheaths will occupy a minimal space, that is have a very low profile in the body lumen. The ability to collapse and assume such a low profile is particularly advantageous in blood vessels in order to reduce the risk of thrombosis and minimize vessel occlusion.

Optionally, a catheter which is usually but not necessarily non-collapsible, may be introduced through the collapsible sheath, typically through a hemostatic valve attached to the sheath. The catheter will usually have at least one lumen for aspirating and/or introducing a fluid or other material, optionally having two or more lumens. Catheters used for aspiration will be non-collapsible (or have at least one non-collapsible lumen) to withstand a vacuum applied to the lumen. A single catheter may be placed within the sheath for extended periods, and optionally two or more catheters may be placed into the sheath, depending on the relative sizes of the sheath and the catheter. The use of the sheaths in accordance with the principles of the present invention reduces trauma and other risks associated with the exchange of access catheters within the sheath.

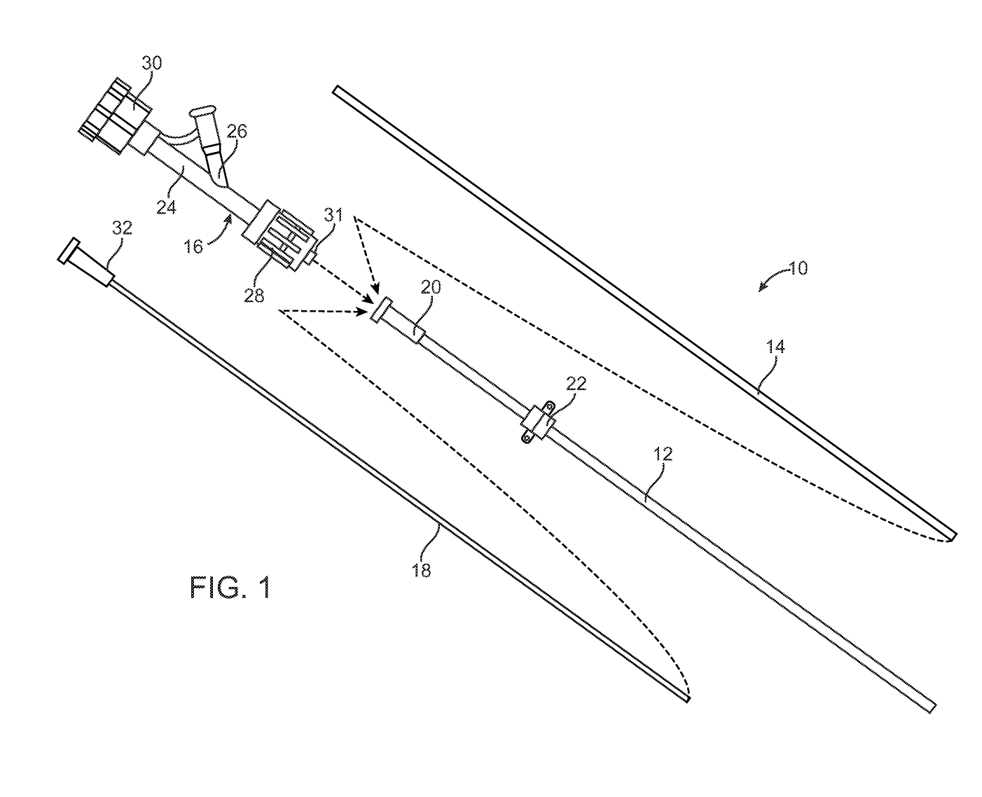

Referring now to FIG. 1, an exemplary percutaneous access system 10 comprises a thin-walled, collapsible sheath 12, an introducer 14, a hemostatic connector 16, and an access catheter 18. The sheath 12 includes both a proximal connector 20 and a tissue anchor 22, illustrated as suture wings in FIG. 1. At least a portion of the sheath 12 distal to the anchor 22 will be collapsible and formed from the materials and having the dimensions generally described above. A portion of the sheath 12 proximal to the connector 22, that is between the tissue anchor 22 and the proximal connector 20, may also be collapsible but could also be non-collapsible and need not have the same structure or properties of the distal portions of the sheath 12. It will be appreciated that the proximal portions of the sheath 12 will at all times be maintained externally to the patient and will therefore will not need to be collapsible to provide the benefits intended by the sheath of the present invention.

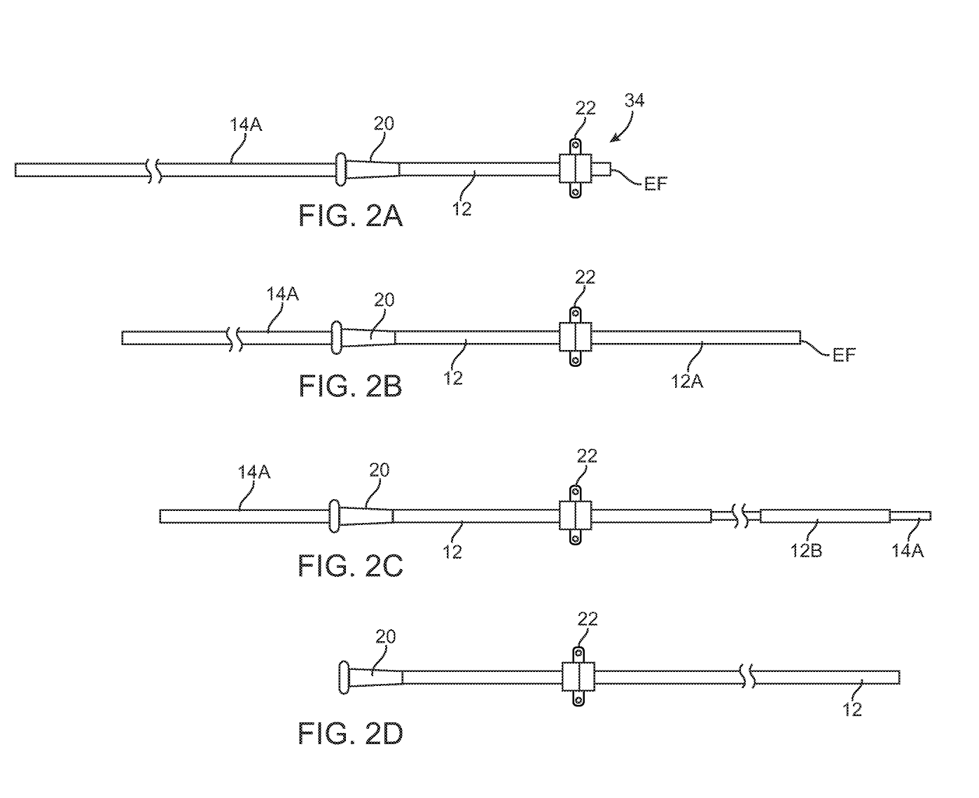

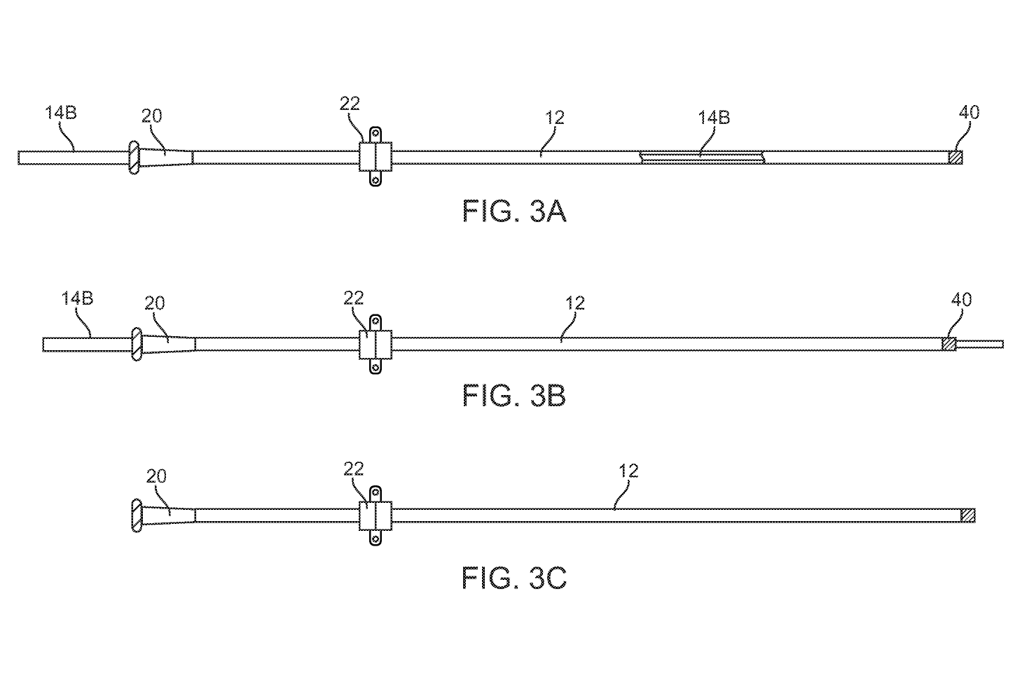

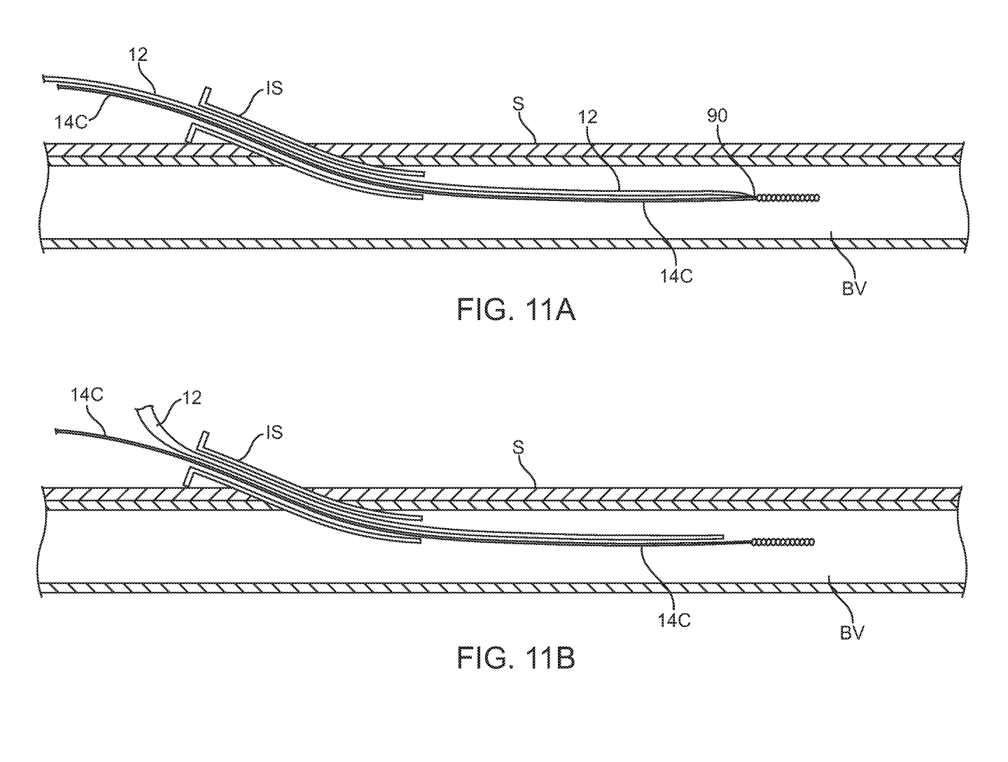

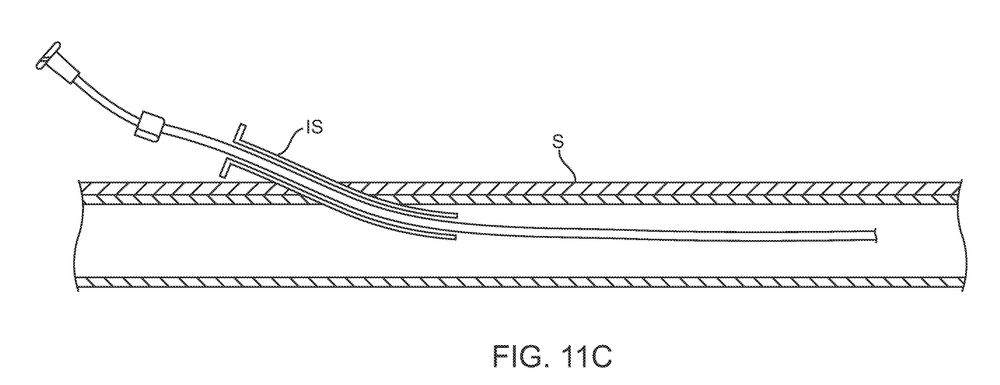

The introducer 14 may be a tubular or solid-core elongate member, as generally described above, or alternatively can be a tubular pusher for everting delivery of the sheath 12, also as described above. Use of a pusher tube will be described in more detail in connection with FIGS. 2A and 2D and FIGS. 10A-10F. Use of an elongate tube for introducing the sheath will be described in connection with FIGS. 3A-3C and FIGS. 11A-11C.

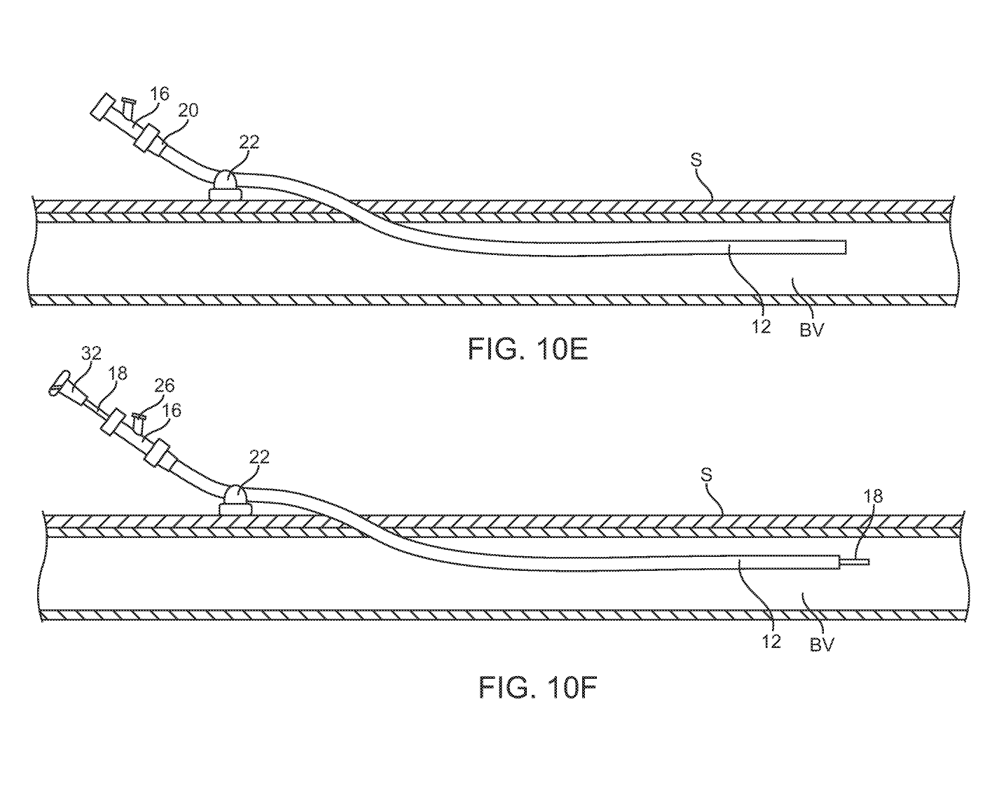

A hemostatic connector 16 preferably comprises a main branch 24 which connects hemostatic valve 30 to an interior connector 31 received in the proximal connector 20 of the sheath 12. Thus, access catheter 18 or other elongate device may be introduced through the hemostatic valve and coaxially within the lumen of the sheath 12. The hemostatic valve allows the user to form a hemostatic seal over the catheter to prevent or minimize blood loss. The side branch 26 of connector 16 also opens into the interior of the sheath 12 through the proximal connector 20. The opening is generally coaxially over any catheter or other element which is introduced through the hemostatic valve 30 so that a separate annular lumen is formed within the lumen of the sheath 12, as described in more detail below in connection with FIGS. 9A-9D.

The access catheter 18 will typically have at least one central lumen, optionally having two or more lumens running axially from at least one connector 32 to openings at or near the distal end. When there are multiple interior lumens, it will usually be desirable to provide a connector 32 having multiple connections. As illustrated, connector 32 is a conventional luer for connection to a single fluid or aspiration source for introducing or aspirating materials from the blood vessel or other body lumen.

Referring now to FIGS. 2A-2D, introducer 14 in the form of a pusher tube 14A may be used to advance the distal portion of sheath 12 into the target body lumen. Initially, as shown in FIG. 2A, the distal portion of sheath 12 is everted proximally into the interior lumen (not shown) of the pusher tube 14A. An everting fold EF is thus created at the distal end 34 of the combination of pusher tube 14A and sheath 12, as shown in FIG. 2A. As the pusher tube 14A is advanced distally, as shown in FIG. 2B, the everting fold advances distally, thus exposing and elongating the distal portion of the sheath 12, where the advanced distal portion is shown as shown as 12A in FIG. 2B. As the pusher tube 14A continues to be distally advanced, the distal portion of sheath 12, shown as 12B in FIG. 2C, is fully deployed and the distal end of the pusher tube 14A emerges from the open end of the sheath. Usually, the pusher tube will then be removed from the sheath 12, leaving the sheath 12 deployed and having an open internal lumen for receiving an access catheter, fluid to be introduced, or other desired purpose. Optionally, the pusher tube 14A could be left in place to provide an access catheter or other therapeutic or diagnostic catheter. When used as an access catheter, the pusher tube 14A could optionally be provided with a luer or other connector (not shown) at its proximal end.

Referring now to FIGS. 3A-3C, an elongate member 14B may also be used as an introducer for advancing the sheath 12 into a target blood vessel or other body lumen. The elongate member 14B is disposed within the central lumen of collapsible sheath 12 and is frangibly attached to the distal end, typically using an adhesive, heat seal, attachment ring, or other attachment mechanism 40. The elongate member 14B can be detached from the attachment mechanism 40, for example by axially advancing the distal end beyond the attachment member 40, as shown in FIG. 3B. Other detachment mechanisms could also be deployed, as generally described above. The after the sheath has been deployed, the elongate member 14B will usually be removed, leaving the collapsible sheath 12 in place and having an open central lumen for use. Optionally, elongate member 14B could be hollow or otherwise have a lumen, allowing it to be used as an access catheter within the sheath 12, in which case the sheath and elongate member could remain attached and the elongate member in place, as long as the lumen of the sheath is not fully occluded and the profile of the elongate member 14B was acceptable when the sheath was collapsed. The hollow lumen is also useful for delivering the combination of sheath and access member over a guidewire.

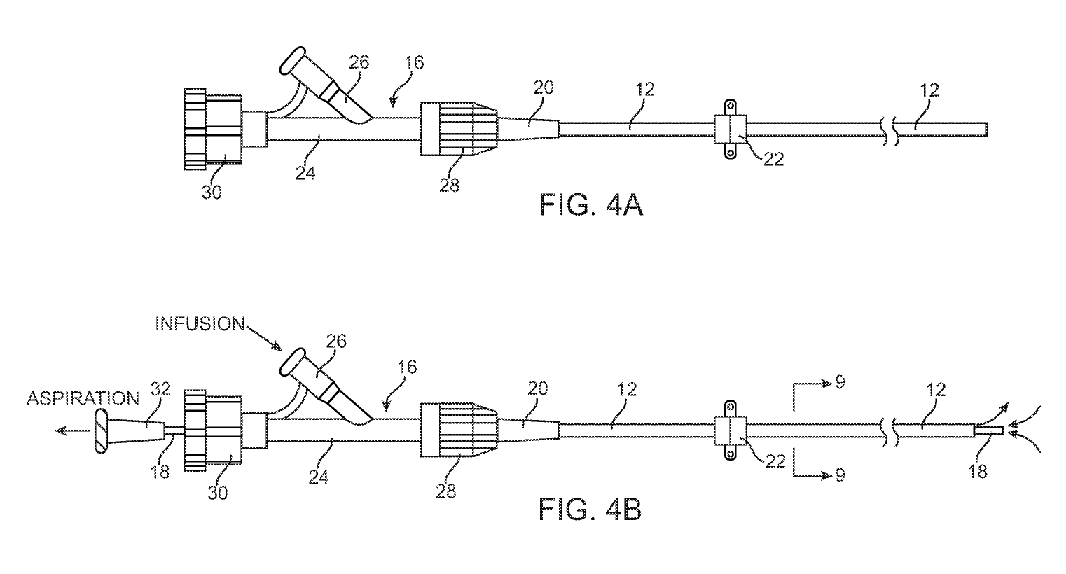

Referring now to FIG. 4A and FIG. 4B, regardless of how the sheath 20 is introduced (using either a pusher tube or elongate member), it will frequently be desirable to connect the hemostatic connector 16 to the proximal connector 20 on the sheath 12. The connector 28 may be attached to the connector 20 in a conventional manner. The main branch 24 of the connector allows access catheter 18 to be introduced through the central lumen of sheath 12, as shown in FIG. 4B. The side branch 26 allows materials to be infused through the annular lumen formed about the exterior of the access catheter 18 within the lumen of sheath 12.

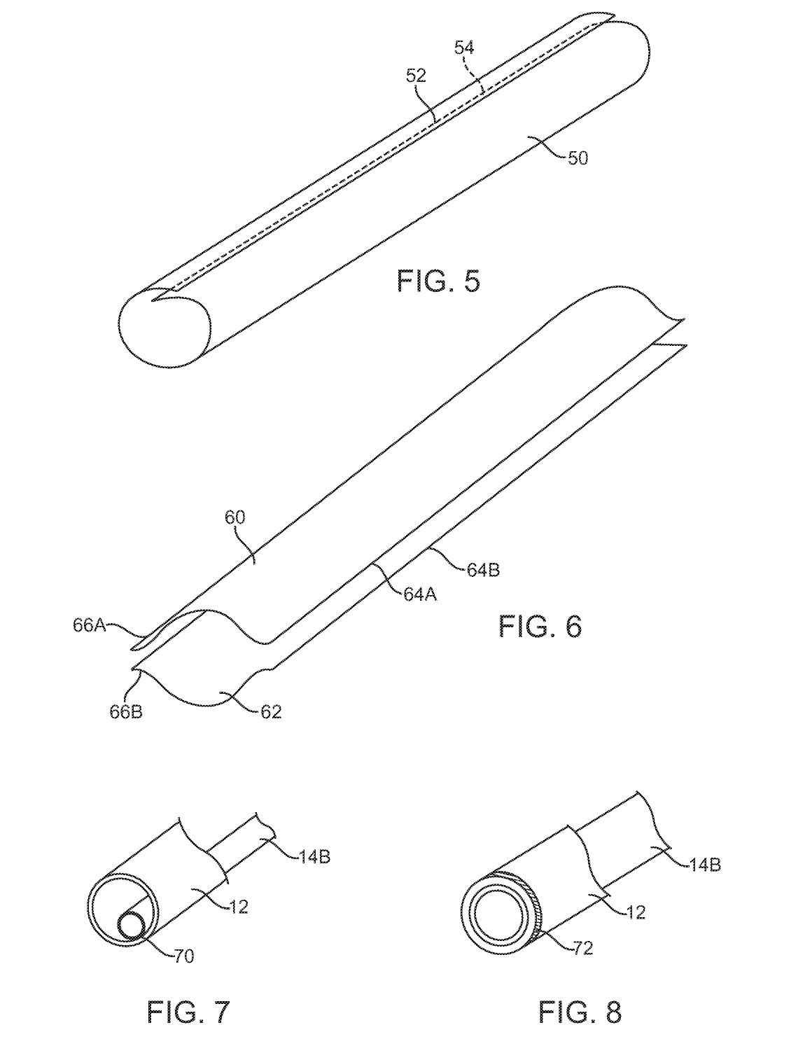

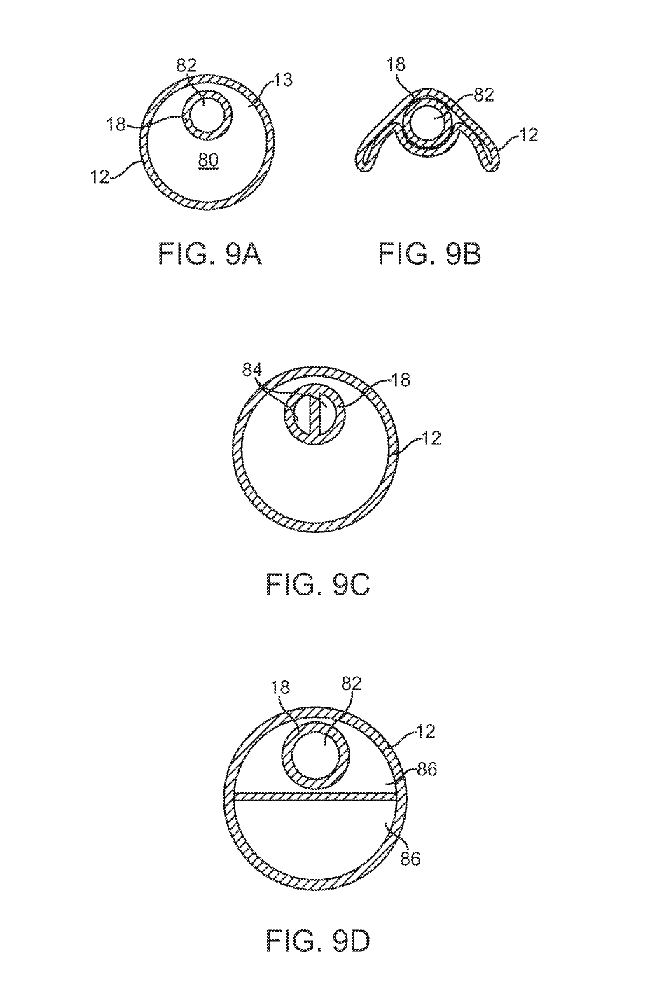

Referring now to FIGS. 5 and 6, the collapsible sheaths will typically be formed from a polymeric material, with exemplary polymers having been listed above. A single thin sheet 50 may be rolled and joined along overlapping edges 52 and 54 to form a single axial seam, as shown in FIG. 5. Alternatively, a pair of sheets 60 and 62 may be joined along edges 64A and 64B, and 66A and 66B, to form the desired tubular structure. The sheets will usually not have any pre-defined or pre-set shape so that they may collapse to a generally flat configuration when the exterior pressure exceeds the interior pressure. Conversely, when a fluid or other material is introduced to raise the interior pressure, the sheath structures will expand to a generally circular cross-section. It is possible that two or more tubular sheaths may be axially attached to form a single multi-lumen sheath or sheath assembly. By joining sheaths with different properties, the resulting structure may be asymmetric when filled with infusion fluid. Likewise, joining adjacent edges of two or more films or sheets having dissimilar widths can create a structure that will expand upon pressurization to a non-circular cross-section, particularly if one or more of the sheets or films is stiffer or thicker than the others so that different portions of the sheath have different flexibilities upon expansion.

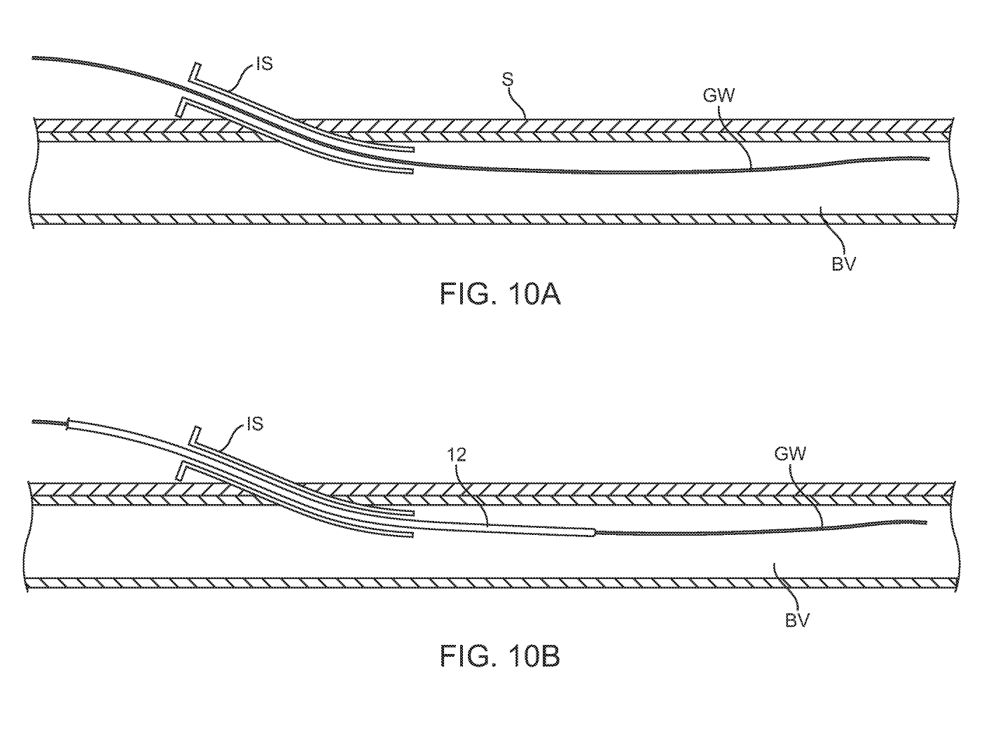

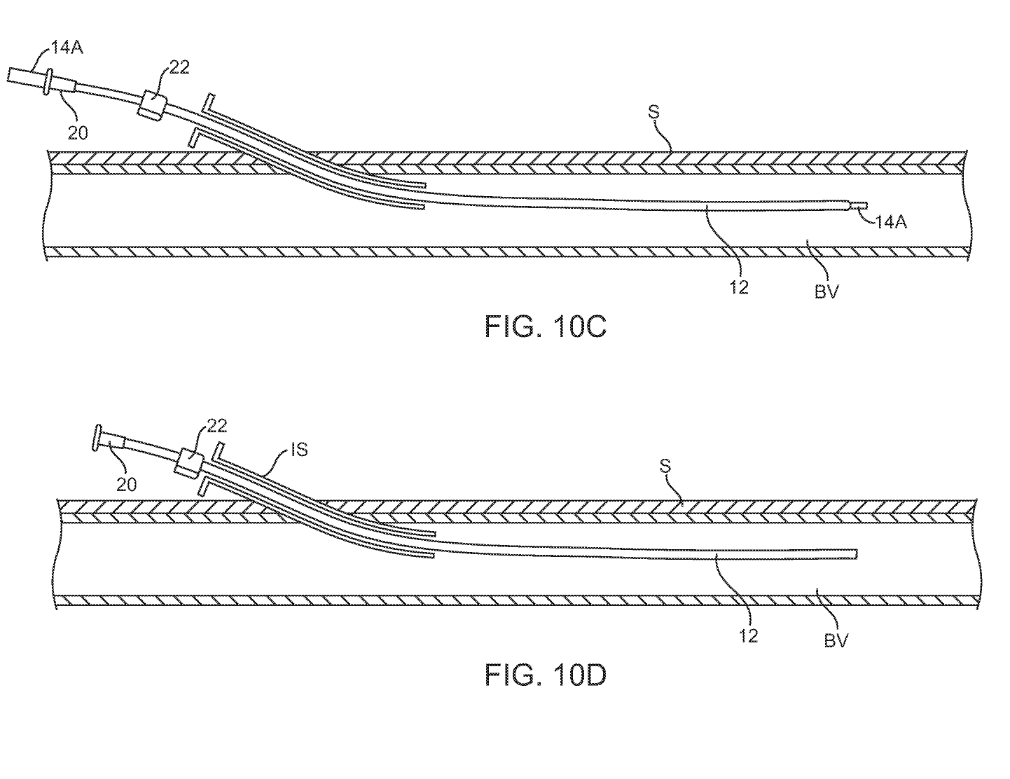

Referring now to FIGS. 7 and 8, elongate members 14B may be joined at the distal ends of sheaths 12 in a variety of ways. For example, as shown in FIG. 7, the elongate member 14B may have a diameter substantially less than that of the interior diameter of the sheath 12. A distal tip of the member 14B may be attached by adhesives or other means at a single point 70 under the distal tip, allowing the sheath to be folded or otherwise collapse over the member 14B as it is being introduced. Alternatively, the elongate member 14B may have outside diameter which is generally the same as the inside diameter of the sheath 12, allowing an attachment ring 72 to be disposed circumferentially around the distal end of the sheath to provide the desired frangible attachment. The ring 72 may optionally be designed to remain on the distal end of the sheath 12 even after the member 14B is withdrawn. Such a ring structure will hold the distal end of the sheath open even when the sheath is not pressurized when the proximal portions of the sheath will collapse in response to vessel pressure. Such an enlarged distal end of the sheath allows blood flow to continually apply an elongation or tensile force along the length of the sheath to keep the sheath extended, where the enlarged portion acts like a balloon in a flow-directed catheter.

Referring now to 9A, when the access catheter 18 is disposed within a central lumen 13 of the sheath 12, a relatively large annular space 80 remains for introducing fluids. As shown in FIG. 9A, a fluid is being introduced through the space 80, thus inflating the sheath 12. When infusion is stopped, and external luminal pressures are present on the exterior sheath 12, the sheath will generally collapse over the access catheter 18, as shown in FIG. 9B. Usually, the access catheter 18 will have at least a single non-collapsible lumen 82, as shown in FIGS. 9A and 9B, which lumen remains open even when the sheath has collapsed. Alternatively, the access catheter 18 may have two or more lumens 84, as shown in FIG. 9C. Additionally or alternatively, the sheath 12 may have two or more lumens 86, as shown in FIG. 9D. The two or more lumens 86 of the sheath may be independently inflated with different infusion media, or in some cases with the same infusion medium.

Referring now to FIGS. 10A-10F, the thin-walled, collapsible sheath 12 can be introduced using pusher tube 14A over a guidewire GW. Access to blood vessel BV may be established using the modified Seldinger technique, or other conventional method, to place an introducer sheath 12 in place, as shown in FIG. 10A. The guidewire may be advanced to a target site within the blood vessel through the introducer sheath 12. The pusher tube 14 is used to advance the sheath 12 by eversion, until a distal end of the pusher tube 14A emerges from the sheath 12, as shown in FIG. 10C. Usually, the pusher tube 14A is then withdrawn, as shown in FIG. 10D, leaving the slack unsupported sheath 12 in place. The introducer sheath 12 may then be withdrawn, and the tissue anchor attached to the skin S, as shown in FIG. 10E. After attaching hemostatic connector 16, the access catheter 18 may be introduced, as shown in FIG. 10F. The combination of sheath 12 and catheter 18 is now available for both introducing pressurized fluids through the side branch 26 of the connector 16, as well as aspirating or infusing materials through the lumen of the access catheter 18.

The thin-walled, collapsible sheath 12 may also be introduced using an elongate member in the form of a guidewire 14C, as shown in FIGS. 11A-11C. The sheath 12 may be frangibly attached at point 90 to the guidewire-type elongate member 14C, as shown in FIG. 11A. After advancing the member 14C to the desired target region with the blood vessel BV, the guidewire may be detached by axially restraining the sheath 12 and relatively advancing the guidewire member 14C, as shown in FIG. 11B. The sheath 12 is then in place, as shown in FIG. 11C, and ready for attachment and use as shown above in FIGS. 10E and 10F.

It will also be possible to attach the sheath to the outside of an elongate member by tightly wrapping, furling, or otherwise conforming the sheath onto the member. The sheath may be uniformly adhered along the sheath's entire length to the elongate member or alternatively may be adhered only at one or more spaced-apart locations. Biologically compatible adhesives may be used to help furl or roll the sheath onto the elongate member and/or attach a portion of the interior surface of the sheath to the elongate member. In such cases, the sheath may be removed from the elongate member by pressurization, rotation of the elongate member relative to the sheath, solubilization of adhesives (if any), upon exposure to blood or other body fluids, and/or to body temperature, or the like.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A method for establishing luminal access, said method comprising:
percutaneously introducing a thin-walled, collapsible sheath through tissue into a body lumen, wherein the sheath has an internal area when expanded;
positioning a non-collapsible catheter within a lumen of the sheath, wherein the catheter has a cross-sectional area less than the internal area of the sheath.

2. A method as in claim 1, wherein the cross-sectional area of the catheter is less than 50% of the luminal area of the sheath.

3. A method as in claim 1, further comprising infusing a fluid through the sheath into the body lumen, wherein at least a portion of the sheath expands to substantially fill the cross-section of the body lumen.

4. A method as in claim 3, further comprising stopping the infusion which allows the sheath to collapse to leave the cross-section of the body lumen substantially open.

5. A method as in claim 1, wherein the body lumen is a blood vessel.

6. A method as in claim 5, wherein the blood vessel is an artery.

7. A method as in claim 5, wherein the blood vessel is a vein.

8. A method as in claim 5, wherein the sheath is positioned through an introducer sleeve passing through skin to the blood vessel.

9. A method as in claim 1, wherein the body lumen is a peritoneal cavity, a nasal sinus, or an intestine.

10. A method as in claim 1, wherein introducing comprises everting the sheath from a distal end of a pusher tube as the pusher tube is advanced through the tissue and into the body lumen.

11. A method as in claim 10, wherein the pusher tube is advanced beyond a distal end of the sheath to release the sheath.

12. A method as in claim 10, wherein the pusher tube is advanced over a guidewire.

13. A method as in claim 10, wherein the sheath radially dilates the tissue as it passes through the tissue.

14. A method as in claim 10, further comprising removing the pusher tube from the sheath prior to introducing the catheter.

15. A method as in claim 10, wherein the pusher tube is left in place as the non-collapsible catheter.

16. A method as in claim 10, wherein a second tube is introduced through the sheath while a first tube remains in place.

17. A method as in claim 1, wherein introducing comprises advancing an elongate member attached to a distal end of the sheath.

18. A method as in claim 17, wherein the elongate member is released from the sheath after the distal end of the sheath is at a target location in the body lumen.

19. A method as in claim 18, wherein the elongate member is advanced while movement of the sheath is constrained to detach the member from the sheath.

20. A method as in claim 18, wherein the elongate member is torqued to release the sheath.

21. A method as in claim 18, wherein a detachment tool is advanced over the elongate member to detach the sheath.

22. A method as in claim 18, wherein the distal end of the sheath enlarges upon release to act as a flow directed catheter.

23. A method as in claim 17, wherein the elongate member is disposed within the sheath as the member is advanced.

24. A method as in claim 23, wherein the sheath is wrapped closely over the elongate member as the elongate member is advanced.

25. A method as in claim 17, wherein the elongate member is disposed along side the sheath as the sheath is advanced.

26. A method as in claim 17, wherein the elongate member comprises a solid core wire.

27. A method as in claim 26, wherein the elongate member comprises a guidewire.

28. A method as in claim 17, wherein the elongate member is a tubular elongate member having an axial passage.

29. A method as in claim 28, wherein the elongate member is advanced over a guidewire.

30. A method as in claim 1, wherein positioning the catheter comprises advancing the catheter through a lumen of the sheath after the sheath has been positioned in the body lumen.

31. A method as in claim 30, wherein a distal end of the catheter is advanced distally beyond a distal end of the sheath.

32. A method as in claim 30, further comprising advancing a second catheter into the sheath.

33. A method as in claim 1, wherein the sheath includes only a single lumen.

34. A method as in claim 1, wherein the sheath has an inner diameter in the range from 1 mm to 12 mm and the catheter has an outer diameter in the range from 0.5 mm to 6 mm.

35. A method as in claim 1, further comprising introducing a material through the annular space to the body lumen.

36. A method as in claim 35, further comprising introducing or aspirating a material to or from the body lumen through a lumen in the catheter.

37. A method for introducing a thin-walled, collapsible sheath and a non-collapsible catheter into a blood vessel, said method comprising:
placing a guidewire in the blood vessel; and
advancing a pusher tube over the guidewire, wherein the sheath everts from a distal end of the pusher tube into the blood vessel lumen.

38. A method for introducing a thin-walled, collapsible sheath and a non-collapsible catheter into a blood vessel, said method comprising:
advancing an elongate member percutaneously into a lumen of the blood vessel, wherein a distal end of the sheath is releasably carried into the blood vessel lumen by the member;
releasing the member from the sheath after the sheath has reached a target location in the blood vessel;
removing the member from the blood vessel leaving the sheath in place; and
introducing the catheter through the sheath into the blood vessel, wherein a flow area is created between an outer surface of the catheter and an inner surface of the sheath.

39. A system for establishing percutaneous access to a body lumen, said system comprising:
a thin-walled sheath having a lumen, a proximal connector, and a skin anchor, said sheath being collapsible over at least a distal portion thereof; and
an introducer adapted to be coupled to the sheath and to advance the sheath within the body lumen.

40. A system as in claim 39, wherein the sheath is collapsible over its entire length.

41. A system as in claim 39, wherein the sheath is non-collapsible over a proximal portion thereof.

42. A system as in claim 41, wherein the proximal portion is reinforced to resist collapse.

43. A system as in claim 39, wherein at least a portion of the sheath is radiopaque.

44. A system as in claim 39, wherein the sheath comprises one or more thin-walled, polymeric tubes.

45. A system as in claim 44, wherein the polymeric tube has a length in the range from 5 cm to 120 cm, an inner diameter in the range from 1 mm to 12 mm, and a wall thickness in the range from 0.01 mm to 0.05 mm.

46. A system as in claim 44, wherein the polymer is a lubricious polymer.

47. A system as in claim 44, wherein the polymer is lubricated.

48. A system as in claim 44, wherein the polymer is selected from the group consisting of polytetrafluoroethylene (PTFE), polyethylene (PE), perfluoroalkoxy (PFA), polyurethane (PU), perfluoromethylvinylether (MFA), perfluoropropylvinylether (PPVE).

49. A system as in claim 48, wherein the polymer comprises tensilized PTFE/PPVE copolymer.

50. A system as in claim 44, wherein the polymeric tube is folded to have a single axial seam.

51. A system as in claim 44, wherein the polymeric tube is formed from two polymeric sheets attached along at least two axial seams.

52. A system as in claim 51, wherein the sheets have different mechanical properties.

53. A system as in claim 44, wherein the sheath has an asymmetric cross-sectional shape.

54. A system as in claim 53, wherein the asymmetric cross-sectional shape is a D-shape.

55. A system as in claim 39, wherein the proximal connector is a luer fitting.

56. A system as in claim 39, wherein the skin anchor comprises suture wings.

57. A system as in claim 39, wherein the skin anchor comprises an adhesive strip.

58. A system as in claim 39, further comprising a hemostatic connector which removably attaches to the proximal connector of the sheath.

59. A system as in claim 58, wherein the hemostatic connector has an axial branch for receiving a catheter to pass coaxially through the sheath and at least a second branch for accessing a flow area between an outside wall of the catheter and an inside wall of the sheath.

60. A system as in claim 58, wherein the flow area is large enough to receive two or more catheters.

61. A system as in claim 39, wherein the introducer comprises an elongate member having a proximal end and a distal end, wherein the member is positioned in the lumen of the sheath and a distal end of the sheath is frangibly connected to a location on the member near its distal end.

62. A system as in claim 61, wherein the distal end of the sheath is adapted to stay open after breaking from the elongate member.

63. A system as in claim 62, wherein the open distal end is adapted to act as a flow direction element in a blood vessel.

64. A system as in claim 61, wherein the member comprises a vascular guidewire having a distal steerable tip, wherein the sheath is connected proximal to the distal tip.

65. A system as in claim 61, wherein the member comprises a tubular member having a central lumen.

66. A system as in claim 65, wherein the central lumen is adapted to receive a guidewire.

67. A system as in claim 61, wherein the introducer comprises a pusher tube having a central lumen, wherein a distal portion of the sheath is disposed within the central lumen of the pusher tube so that the sheath will evert from the distal portion as the pusher tube is advanced through the body lumen.

68. A system as in claim 39, further comprising a catheter adapted to be introduced through the proximal connector and into the lumen of the sheath.

69. A system as in claim 68, wherein the catheter has an outside diameter which is smaller than an inside diameter of the sheath to leave an annular lumen when the catheter is within the sheath lumen.

70. A system as in claim 69, wherein the catheter has an outside diameter in the range from 0.5 mm to 6 mm and the sheath has an inside diameter in the range from 1 mm to 12 mm.

71. A system as in claim 70, wherein an annular lumen having a width in the range from 0.5 mm to 2 mm is formed when the catheter is present in the lumen of the sheath.
